(19) United States
(12) Patent Application Publication
(10) Pub. No.: US 2009/0253905 A1
(43) Pub. Date: Oct. 8, 2009

(54) PROCESS FOR THE PREPARATION OF ONIUM SALTS WITH ALKYL- OR ARYLSULFONATE ANIONS OR ALKYL- OR ARYLCARBOXYLATE ANIONS HAVING A LOW HALIDE CONTENT

(76) Inventors: Nikolai (Mykola) Ignatyev, Duisburg (DE); Urs Weiz-Biermann, Heppenheim (DE); Andriy Kucheryna, Kiev (UA); Helge Willner, Muelheim/Ruhr (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/721,617

(22) PCT Filed: Nov. 18, 2005
(86) PCT No.: PCT/EP05/12401

§ 371 Date: Jun. 13, 2007

(30) Foreign Application Priority Data
Dec. 14, 2004 (DE) 10 2004 060 076.7

(51) Int. Cl. C07D 265/30; C07D 233/54; C07D 213/20; C07D 295/02; C07F 7/02; C07D 249/04; C07D 239/02; C07D 237/02; C07D 241/02; C07D 277/20; C07D 263/02; C07D 231/02; C07D 249/08

(52) U.S. Cl. 544/109; 548/335.1; 546/347; 548/579; 548/406; 548/110; 546/14; 548/255; 544/242; 544/224; 544/336; 548/146; 548/215; 548/356.1; 548/262.2

(57) ABSTRACT

The invention relates to a method for producing onium salts comprising alkyl anions or aryl sulfonate anions or alkyl carboxylate anions or acryl carboxylate anions by reacting an onium halide with an alkyl silyl ester or trialkyl silyl ester of an alkyl sulfonic acid or aryl sulfonic acid or an alkyl carboxylic acid or aryl carboxylic acid or the anhydrides thereof.

The invention relates to a process for the preparation of onium salts with alkyl- or arylsultfonate anions or alkyl- or arylcarboxylate anions by reaction of an onium halide with an alkyl or trialkylsilyl ester of an alkyl- or aryl-sulfonic acid or alkyl- or arylcarboxylic acid or anhydrides thereof.

A large number of onium salts, in particular alkyl- or arylsulfonates or alkyl- or arylcarboxylates, are ionic liquids. Owing to their properties, ionic liquids represent an effective alternative to traditional volatile organic solvents for organic synthesis in modern research. The use of ionic liquids as novel reaction medium could furthermore be a practical solution both for solvent emission and also for problems in the reprocessing of catalysts.

Ionic liquids or liquid salts are ionic species which consist of an organic cation and a generally inorganic anion. They do not contain any neutral molecules and usually have melting points below 373 K. However, the melting point may also be higher without restricting the usability of the salts in all areas of application. Examples of organic cations are, inter alia, tetraalkylammonium, tetraalkylphosphonium, N-alkylpyridinium, 1,3-dialkylimidazolium or trialkylsulfonium. Amongst a multiplicity of suitable anions, mention may be made, for example, of BF₄⁻, PF₆⁻, SbF₆⁻, NO₃—, CF₃SO₃—, (CF₃SO₂)₂N⁻, arylSO₃⁻, CF₃CO₂⁻, CH₃CO₂⁻ or Al₂Cl7⁻.

A general method for the preparation of onium sulfonates or carboxylates is alkylation of the organic base, i.e., for example, the amine, phosphine, guanidine or heterocyclic base, using alkyl esters of an alkyl- or arylsulfonic acid, for example alkyl p-toluenesulfonate (alkyl tosylate), or of an alkyl- or arylcarboxylic acid, for example alkyl trifluoroacetate, also disclosed by (Richard C. Larock, Comprehensive Organic Transformations, 2nd Edition, Wiley-VCH, New York, 1999, or P. Wasserscheid and T. Welton (Eds), Ionic Liquids in Synthesis, Wiley-VCH, 2003).

A disadvantage of this method is, however, that a substituent of the onium cation formed always corresponds to the corresponding alkyl group of the alkyl ester. If, for example, 1-butylimidazolium is reacted with methyl p-toluenesulfonate, 1-butyl-3-methylimidazolium p-toluenesulfonate is formed. However, asymmetrically substituted onium salts, i.e. salts in which the alkyl group of the ester employed is not a substituent of the onium salt formed, are desired.

Asymmetrical onium salts with alkyl- or arylsulfonate anions or alkyl- or arylcarboxylate anions, as defined above, can also be prepared by a metathesis by reacting an onium halide with a corresponding alkali metal salt of the corresponding acid. However, the alkali metal halide formed, for example sodium chloride, has to be removed by an additional purification method. The contamination by halide ions, for example chloride ions, greater than 1000 ppm (0.1%), reduces the usability of the ionic liquid, in particular in the use for electrochemical processes. The technology is therefore of crucial importance in processes for the preparation of onium salts, in particular ionic liquids, in order that they can be synthesised with low impurity levels by the reaction per se or by the reaction procedure, and thus further expensive additional process steps during the synthesis are superfluous.

The object of the present invention was accordingly to provide an alternative process for the preparation of onium salts with alkyl- or arylsulfonate anions or alkyl- or arylcarboxylate anions having a low halide content which results in salts, preferably of asymmetrically substituted onium salts, of high purity in good yield and is also suitable for large-scale industrial production.

A process of this type is of course then also suitable for the preparation of symmetrically substituted onium sulfates.

The object is achieved by the process according to the invention since the alkyl or trialkylsilyl ester of the sulfonic or carboxylic acid employed or the corresponding anhydride alkylates or acylates the anion of the onium halide employed and not the organic onium cation. The alkyl, trialkylsilyl or acyl halides formed as by-product are generally gases or very volatile compounds which can be removed from the reaction mixture without major engineering effort. Some of these by-products are themselves valuable materials for organic syntheses.

The invention therefore relates to a process for the preparation of onium salts with alkyl- or arylsulfonate anions or alkyl- or arylcarboxylate anions by reaction of an onium halide with an alkyl or trialkylsilyl ester of an alkyl- or arylsulfonic acid or alkyl- or arylcarboxylic acid or anhydrides thereof. In accordance with the invention, the reaction of the onium halide with an alkyl ester of a sulfonic acid is carried out at room temperature.

U.S. Pat. No. 2,585,979 discloses the preparation of quaternary sulfonates of pyrimidylaminoquinoline derivatives

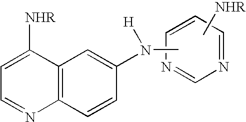

by reaction of the corresponding pyrimidylaminoquinoline halide with a lower-alkyl ester of a sulfonic acid. However, the reaction with methyl methanesulfonate, in contrast to the process according to the invention, is carried out at temperatures of 130° to 140° C. in the presence of a solvent, for example a mixture of nitrobenzene and toluene. The reaction with methyl p-toluenesulfonate is carried out without addition of solvent, likewise at high temperatures of 130° to 135° C. Surprisingly, however, the reaction according to the invention with the alkyl esters of sulfonic acids succeeds at room temperature, i.e. at temperatures between 10° and 30° C., without the use of a solvent, in approximately quantitative yield.

U.S. Pat. No. 2,585,979 contains no indication that trialkylsilyl esters of the sulfonic or carboxylic acid or an anhydride of the sulfonic or carboxylic acid could be employed.

However, the process is particularly distinguished on use of trialkylsilyl esters of alkyl- or arylsulfonic or -carboxylic acids since these compounds have low toxicity.

The trialkylsilyl esters of alkyl- or arylsulfonic acids are preferably suitable for the preparation of the onium salts with alkyl- or arylsulfonate anions by the process according to the invention.

The anhydrides of alkyl- or arylcarboxylic acids are preferably suitable for the preparation of the onium salts with alkyl- or arylcarboxylate anions by the process according to the invention.

Suitable onium halides are ammonium halides, phosphonium halides, thiouronium halides, guanidinium halides or halides with a heterocyclic cation, where the halides can be selected from the group chlorides, bromides or iodides. Chlorides or bromides are preferably employed in the process according to the invention. Iodides are preferably employed for the synthesis of the thiouronium salts.

The onium halides are generally commercially available or can be prepared by synthetic methods as known from the literature, for example in the standard works, such as Houben-Weyl, Methoden der organischen Chemie [Methods of Organic Chemistry], Georg-Thieme-Verlag, Stuttgart, Richard C. Larock, Comprehensive Organic Transformations, 2nd Edition, Wiley-VCH, New York, 1999, or P. Wasserscheid, T. Welton (Eds), Ionic Liquids in Synthesis, Wiley-VCH, 2003. Use can also be made here of variants known per se which are not mentioned here in greater detail.

Ammonium halides can be described, for example, by the formula (1)

[NR₄]⁺Hal⁻ (1),

phosphonium halides can be described, for example, by the formula (2)

[PR₄]+Hal⁻ (2),

where
Hal denotes Cl, Br or I and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms,
which may be substituted by alkyl groups having 1-6 C atoms,
where one or more R may be partially or fully substituted by —F, but where all four or three R must not be fully substituted by F,
and where, in the R, one or two non-adjacent carbon atoms which are not in the α- or ω-position may be replaced by atoms and/or atom groups selected from the group —O—, —S—, —S(O)— or —SO₂—.

However, compounds of the formulae (1) and (2) in which all four or three substituents R are fully substituted by halogens, for example tris(trifluoromethyl)methylammonium chloride, tetra(trifluoromethyl)ammonium chloride or tetra(nonafluorobutyl)ammonium chloride, are excluded.

Thiouronium halides can be described, for example, by the formula (3)

[(R¹R²N)—C(═SR⁷)(NR³R⁴)]+Hal⁻ (3)

and guanidinium halides can be described, for example, by the formula (4)

[C(NR¹R²)(NR³R⁴)(NR⁵R⁶)]⁺Hal⁻ (4),

where
Hal denotes Cl, Br or I and
R¹ to R⁷ each, independently of one another, denote
hydrogen or CN, where hydrogen is excluded for R⁷,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms,
which may be substituted by alkyl groups having 1-6 C atoms,
where one or more of the substituents R¹ to R⁷ may be partially or fully substituted by —F, but where all substituents on an N atom must not be fully substituted by F,
where the substituents R¹ to R⁷ may be connected to one another in pairs by a single or double bond
and where, in the substituents R¹ to R⁷, one or two non-adjacent carbon atoms which are not bonded directly to the heteroatom and are not in the ω-position may be replaced by atoms and/or atom groups selected from the group —O—, —S—, —S(O)— or —SO₂—.

Halides with a heterocyclic cation can be described, for example, by the formula (5)

[HetN]⁺Hal⁻ (5),

where
Hal denotes Cl, Br or I and
HetN⁺ denotes a heterocyclic cation selected from the group

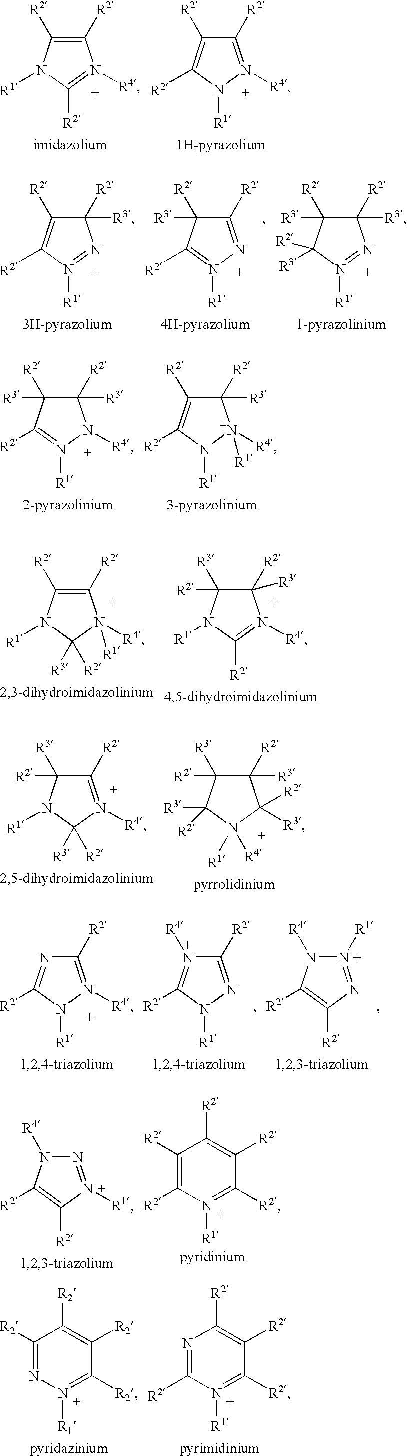

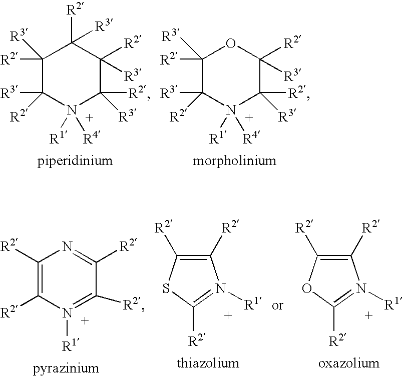

where the substituents
R¹′ to R⁴′ each, independently of one another, denote
hydrogen or CN,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
dialkylamino having alkyl groups having 1-4 C atoms, but which is not bonded to the heteroatom of the heterocycle,
saturated, partially or fully unsaturated cycloalkyl having 3-7 C atoms,
which may be substituted by alkyl groups having 1-6 C atoms, or aryl-C₁-C₆-alkyl,
where the substituents R^{1′} and R⁴′ may be partially or fully substituted by F, but where R^{1′} and R^{4′} are not simultaneously CN or must not simultaneously be fully substituted by F,
where the substituents R^{2′} and R^{3′} may be partially or fully substituted by halogens or partially substituted by NO₂ or CN
and where, in the substituents R¹′ to R⁴′, one or two non-adjacent carbon atoms which are not bonded directly to the heteroatom and are not in the ω-position may be replaced by atoms and/or atom groups selected from the group —O—, —S—, —S(O)— or —SO₂—.

For the purposes of the present invention, fully unsaturated substituents are also taken to mean aromatic substituents.

In accordance with the invention, suitable substituents R and R¹ to R⁷ of the compounds of the formulae (1) to (4), besides hydrogen, are preferably: C₁- to C₂₀-, in particular C₁- to C₁₄-alkyl groups, and saturated or unsaturated, i.e. also aromatic, C₃- to C₇-cycloalkyl groups, which may be substituted by C₁- to C₆-alkyl groups, in particular phenyl. However, the substituents R and R¹ to R⁷ may likewise be substituted by further functional groups, for example by CN, SO₂R′, SO₂OR′ or COOR′. R′ denotes non- or partially fluorinated C₁- to C₆-alkyl, C₃- to C₇-cycloalkyl, unsubstituted or substituted phenyl.

The substituents R in the compounds of the formula (1) or (2) may be identical or different here. Preferably, three substituents in the formulae (1) and (2) are identical and one substituent is different.

The substituent R is particularly preferably methyl, ethyl, isopropyl, propyl, butyl, sec-butyl, pentyl, hexyl, octyl, decyl or tetradecyl.

Up to four substituents of the guanidinium cation [C(NR¹R²)(NR³R⁴)(NR⁵R⁶)]⁺ may also be connected in pairs in such a way that mono-, bi- or polycyclic cations are formed.

Without restricting generality, examples of such guanidinium cations are:

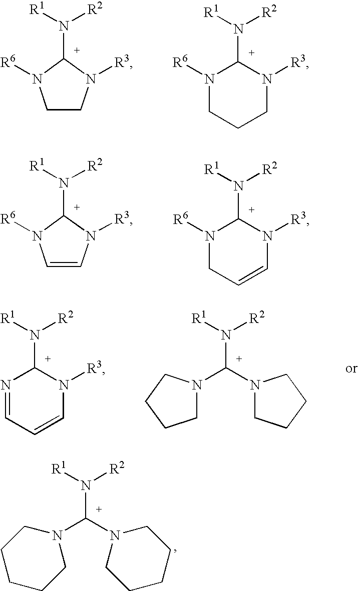

where the substituents R¹ to R³ and R⁶ may have an above-mentioned or particularly preferred meaning.

The carbocycles or heterocycles of the above-mentioned guanidinium cations may optionally also be substituted by C₁- to C₆-alkyl, C₁- to C₆-alkenyl, NO₂, F, Cl, Br, I, C₁-C₆-alkoxy, SCF₃, SO₂CF₃, SO₂CH₃, CN, COOR″, SO₂NR″₂, SO₂X′ or SO₃R″, where X′ denotes F, Cl or Br and R″ denotes a non- or partially fluorinated C₁- to C₆-alkyl or C₃- to C₇-cycloalkyl as defined for R′, or by substituted or unsubstituted phenyl.

Up to four substituents of the thiouronium cation [(R¹R²N)—C(═SR⁷)—(NR³R⁴)]⁺ may also be connected in pairs in such a way that mono-, bi- or polycyclic cations are formed.

Without restricting generality, examples of such cations are indicated below:

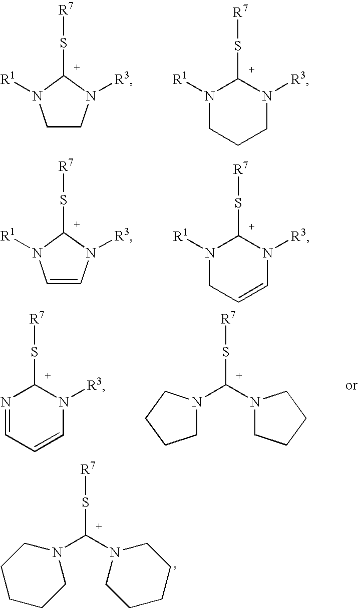

where the substituents R¹, R³ and R⁷ may have an above-mentioned or particularly preferred meaning. The carbocycles or heterocycles of the above-mentioned thiouronium cations may optionally also be substituted by C₁- to C₆-alkyl, C₁- to C₆-alkenyl, NO₂, F, Cl, Br, I, C₁-C₆-alkoxy, SCF₃, SO₂CF₃, SO₂CH₃, CN, COOR″, SO₂NR″₂, SO₂X′ or SO₃R″, where X′ denotes F, Cl or Br and R″ denotes a non- or partially fluorinated C₁- to C₆-alkyl or C₃- to C₇-cycloalkyl as defined for R′, or by substituted or unsubstituted phenyl.

The C₁-C₁₄-alkyl group is, for example, methyl, ethyl, isopropyl, propyl, butyl or sec-butyl, furthermore also pentyl, 1-, 2- or 3-methylbutyl, 1,1-, 1,2- or 2,2-dimethylpropyl, 1-ethylpropyl, hexyl, heptyl, octyl, nonyl, decyl, undecyl, dodecyl, tridecyl or tetradecyl, optionally perfluorinated, for example as difluoromethyl, trifluoromethyl, pentafluoroethyl, heptafluoropropyl or nonafluorobutyl.

A straight-chain or branched alkenyl having 2 to 20 C atoms, where a plurality of double bonds may also be present, is, for example, vinyl, allyl, 2- or 3-butenyl, isobutenyl, sec-butenyl, furthermore 4-pentenyl, isopentenyl, hexenyl, heptenyl, octenyl, —C₉H₁₇, —C₁₀H₁₉ to —C₂₀H₃₉, preferably allyl, 2- or 3-butenyl, isobutenyl, sec-butenyl, furthermore preferably 4-pentenyl, isopentenyl or hexenyl.

A straight-chain or branched alkynyl having 2 to 20 C atoms, where a plurality of triple bonds may also be present, is, for example, ethynyl, 1- or 2-propynyl, 2- or 3-butynyl, furthermore 4-pentynyl, 3-pentynyl, hexynyl, heptynyl, octynyl, —C₉H₁₅, —C₁₀H₁₇ to —C₂₀H₃₇, preferably ethynyl, 1- or 2-propynyl, 2- or 3-butynyl, 4-pentynyl, 3-pentynyl or hexynyl.

Aryl-C₁-C₆-alkyl denotes, for example, benzyl, phenylethyl, phenylpropyl, phenylbutyl, phenylpentyl or phenylhexyl, where both the phenyl ring and also the alkylene chain may be partially or fully substituted as described above by —F, particularly preferably benzyl or phenylpropyl. However, the phenyl ring or also the alkylene chain may likewise be substituted by further functional groups, for example by CN, SO₂R′, SO₂OR′ or COOR′. R′ here has a meaning defined above.

Unsubstituted saturated or partially or fully unsaturated cycloalkyl groups having 3-7 C atoms are therefore cyclopropyl, cyclobutyl, cyclopentyl, cyclohexyl, cycloheptyl, cyclopentenyl, cyclopenta-1,3-dienyl, cyclohexenyl, cyclohexa-1,3-dienyl, cyclohexa-1,4-dienyl, phenyl, cycloheptenyl, cyclohepta-1,3-dienyl, cyclohepta-1,4-dienyl or cyclohepta-1,5-dienyl, each of which may be substituted by C₁- to C₆-alkyl groups, where the cycloalkyl group or the C₁- to C₆-alkyl-substituted cycloalkyl group may in turn also be substituted by F. However, the cycloalkyl groups may likewise be substituted by further functional groups, for example by CN, SO₂R′, SO₂OR′ or COOR′. R′ here has a meaning defined above.

In the substituents R, R¹ to R⁶ or R^{1′} to R^{4′}, one or two non-adjacent carbon atoms which are not bonded in the α-position to the heteroatom or in the ω-position may also be replaced by atoms and/or atom groups selected from the group —O—, —S—, —S(O)— or —SO₂—.

Without restricting generality, examples of substituents R, R¹ to R⁶ and R^{1′} to R^{4′} modified in this way are:

—OCH₃, —OCH(CH₃)₂, —CH₂OCH₃, —CH₂—CH₂—O—CH₃, —C₂H₄OCH(CH₃)₂, —C₂H₄SC₂H₅, —C₂H₄SCH(CH₃)₂, —S(O)CH₃, —SO₂CH₃, —SO₂C₆H₅, —SO₂C₃H₇, —SO₂CH(CH₃)₂, —SO₂CH₂CF₃, —CH₂SO₂CH₃, —O—C₄H₈—O—C₄H₉, —CF₃₁—C₂F₆, —C₃F₇, —C₄F₉, —CF₂CF₂H, —CF₂CHFCF₃, —CF₂CH(CF₃)₂, —C₂F₄N(C₂F₅)C₂F₅, —CHF₂, —CH₂CF₃, —C₂F₂H₃, —C₃H₆, —CH₂C₃F₇, —CH₂C(O)OCH₃, —CH₂C₆H₅ or —C(O)C₆H₅.

R′ is C₃- to C₇-cycloalkyl, for example cyclopropyl, cyclobutyl, cyclopentyl, cyclohexyl or cycloheptyl.

In R′, substituted phenyl denotes phenyl which is substituted by C₁- to C₆-alkyl, C₁- to C₆-alkenyl, NO₂, F, Cl, Br, I, C₁-C₆-alkoxy, SCF₃, SO₂CF₃, SO₂CH₃, COOR″, SO₂X′, SO₂NR″₂ or SO₃R″, where X′ denotes F, Cl or Br and R″ denotes a non- or partially fluorinated C₁- to C₆-alkyl or C₃- to C₇-cycloalkyl as defined for R′, for example o-, m- or p-methylphenyl, o-, m- or p-ethylphenyl, o-, m- or p-propylphenyl, o-, m- or p-isopropylphenyl, o-, m- or p-nitrophenyl, o-, m- or p-methoxyphenyl, o-, m- or p-ethoxyphenyl, o-, m-, p-(trifluoromethyl)phenyl, o-, m-, p-(trifluoromethoxy)phenyl, o-, m-, p-(trifluoromethylsulfonyl)phenyl, o-, m- or p-fluorophenyl, o-, m- or p-chlorophenyl, o-, m- or p-bromophenyl, o-, m- or p-iodophenyl, further preferably 2,3-, 2,4-, 2,5-, 2,6-, 3,4- or 3,5-dimethylphenyl, 2,3-, 2,4-, 2,6-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3-, 2,4-, 2,5-, 2,6-, 3,4- or 3,5-dimethoxyphenyl, 5-fluoro-2-methylphenyl, 3,4,5-trimethoxyphenyl or 2,4,5-trimethylphenyl.

The substituents R¹ to R⁷ are each, independently of one another, preferably a straight-chain or branched alkyl group having 1 to 10 C atoms. The substituents R¹ and R², R³ and R⁴ and R⁵ and R⁶ in compounds of the formulae (3) and (4) may be identical or different here.

R¹ to R⁷ are particularly preferably each, independently of one another, methyl, ethyl, n-propyl, isopropyl, n-butyl, sec-butyl, phenyl or cyclohexyl, very particularly preferably methyl, ethyl, n-propyl, isopropyl or n-butyl.

In accordance with the invention, suitable substituents R^{1′} to R^{4′} of compounds of the formula (5), besides hydrogen, are preferably: CN, C₁- to C₂₀-, in particular C₁- to C₁₂-alkyl groups, and saturated or unsaturated, i.e. also aromatic, C₃- to C₇-cycloalkyl groups, which may be substituted by C₁- to C₆-alkyl groups, in particular phenyl or aryl-C₁-C₆-alkyl.

However, the substituents R^{1′} to R^{4′} may likewise be substituted by further functional groups, for example by CN, SO₂R′, SO₂OR′ or COOP′. R′ denotes non- or partially fluorinated C₁- to C₆-alkyl, C₃- to C₇-cycloalkyl, un-substituted or substituted phenyl.

The substituents R^{1′} and R^{4′} are each, independently of one another, particularly preferably CN, methyl, ethyl, isopropyl, propyl, butyl, sec-butyl, pentyl, hexyl, octyl, decyl, cyclohexyl, phenyl, phenylpropyl or benzyl. They are very particularly preferably methyl, CN, ethyl, n-butyl or hexyl. In pyrrolidinium or piperidinium compounds, the two substituents R^{1′} and R^{4′} are preferably different.

The substituent R^{1′} or R^{4′} is in each case, independently of one another, in particular hydrogen, dimethylamino, diethylamino, methyl, ethyl, isopropyl, propyl, butyl, sec-butyl, cyclohexyl, phenyl or benzyl. R²′ is particularly preferably hydrogen, methyl, ethyl, isopropyl, propyl, butyl, sec-butyl or dimethylamino. R^{2′} and R^{3′} are very particularly preferably hydrogen.

The alkyl groups as substituents R and R¹ to R⁶ and R^{1′} and R^{4′} of the heterocyclic cations of the formula (5) are preferably different from the alkyl group of the alkyl ester of the alkyl- or arylsulfonic acid or alkyl- or arylcarboxylic acid employed.

The onium sulfonate or carboxylate prepared in accordance with the invention may, however, also have alkyl groups in the cation which are identical with the alkyl group in the ester, but were not introduced in accordance with the invention by alkylation. The focus is then on the simple reaction procedure and the particularly low halide content in the end product, as is important for the reaction according to the invention with trialkylsilyl esters of the alkyl or arylsulfonic acids or alkyl- or arylcarboxylic acids or anhydrides thereof.

HetN⁺ of the formula (5) is preferably

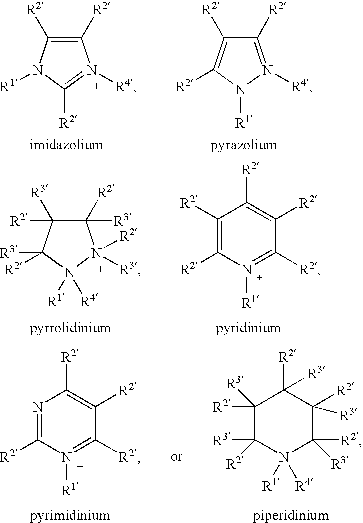

where the substituents R^{1′} to R^{4′} each, independently of one another, have a meaning described above.

HetN⁺ is particularly preferably imidazolium, pyrrolidinium or pyridinium, as defined above, where the substituents R^{1′} to R^{4′} each, independently of one another, have a meaning described above.

The alkyl ester of an alkyl- or arylsulfonic acid employed is preferably an alkyl ester having a straight-chain or branched alkyl group having 1-8 C atoms, preferably having 1-4 C atoms, where the alkyl group may also contain Cl or F atoms. Examples of alkyl esters of sulfonic acids are, for example, methyl trifluoromethanesulfonate, ethyl trifluoromethanesulfonate, propyl trifluoromethanesulfonate, butyl trifluoromethanesulfonate, methyl methanesulfonate, ethyl methanesulfonate, propyl methanesulfonate, butyl methanesulfonate, methyl ethanesulfonate, ethyl ethanesulfonate, propyl ethanesulfonate, butyl ethanesulfonate, methyl benzenesulfonate, ethyl benzenesulfonate, methyl p-toluenesulfonate, ethyl p-toluenesulfonate, propyl p-toluenesulfonate, methyl p-nitrobenzenesulfonate or ethyl p-nitrobenzenesulfonate.

Particular preference is given to the use of methyl trifluoromethanesulfonate, ethyl trifluoromethanesulfonate, methyl methanesulfonate, methyl benzenesulfonate, methyl p-toluenesulfonate or ethyl p-toluenesulfonate. Very particular preference is given to the use of methyl trifluoromethanesulfonate or ethyl trifluoromethanesulfonate.

Alkyl esters or trialkylsilyl esters of chlorosulfonic acid can also be used in the process according to the invention, for example methyl chlorosulfonate or trimethylsilyl chlorosulfonate.

The trialkylsilyl ester of an alkyl- or arylsulfonic acid employed is preferably a trialkylsilyl ester having a straight-chain or branched alkyl group having 1-8 C atoms, preferably having 1-4 C atoms, where each alkyl group is independent of one another. The alkyl groups on the silicon are preferably identical. Trialkylsilyl esters of sulfonic acids are, for example, CF₃—SO₂—O—Si(CH₃)₃, CF₃—SO₂—O—Si(CH₃)₂(t-C₄H₉), CF₃—SO₂—O—Si(C₂H₅)₂(i-C₃H₇), CF₃—SO₂—O—Si(C₂H₆)₃, CF₃—SO₂—O—Si(i-C₃H₇)₃, CF₃—SO₂—O—Si-(n-C₄H₉)₃, CH₃SO₂—O—Si(CH₃)₃, CH₃—SO₂—O—Si(CH₃)₂(n-C₄H₉), (C₂H₅)—SO₂—O—Si—(CH₃)₃, (C₂H₅)—SO₂—O—Si(C₂H₅)₃, C₂F₅—SO₂—O—Si(CH₃)₃, C₄F₉SO₂—O—Si(CH₃)₃, C₄F₉—SO₂—O—Si(C₂H₅)₃, C₄F₉—SO₂—O—Si(CH₃)₂(n-C₄H₉), C₄F₉—SO₂—O—Si(CH₃)(n-C₄H₉)₂, C₈F₁₇—SO₂—O—Si(CH₃)₃, C₆F₅—SO₂—O—Si(CH₃)₃ or (p-CH₃)C₆H₄—SO₂—O—Si(CH₃)₃. Particular preference is given to the use of CF₃—SO₂—O—Si(CH₃)₃, CH₃SO₂—O—Si(CH₃)₃, (C₂H₅)—SO₂—O—Si(C₂H₅)₃, C₄F₉SO₂—O—Si(CH₃)₃, C₈F₁₇—SO₂—O—Si(CH₃)₃, C₆F₅—SO₂—O—Si(CH₃)₃ or (p-CH₃)C₆H₄—SO₂—O—Si(CH₃)₃, very particular preference is given to the use of trimethylsilyl methanesulfonate (CH₃SO₂—O—Si(CH₃)₃), trimethylsilyl pentafluorobenzenesulfonate (C₆F₅SO₂—O—Si(CH₃)₃), trimethylsilyl toluenesulfonate ((p-CH₃)C₆H₄—SO₂—O—Si(CH₃)₃) or trimethylsilyl trifluoromethanesulfonate (CF₃SO₂—O—Si(CH₃)₃).

The alkyl ester of an alkyl- or arylcarboxylic acid employed is preferably an alkyl ester having a straight-chain or branched alkyl group having 1-8 C atoms, preferably having 1-4 C atoms. Alkyl esters of carboxylic acids are, for example, methyl trifluoroacetate, ethyl trifluoroacetate, propyl trifluoroacetate, butyl trifluoroacetate, tert-butyl acetate, tert-butyl propionate, isopropyl benzoate, tert-butyl benzoate, methyl 4-nitrobenzoate or methyl pentafluorobenzoate. Particular preference is given to the use of methyl trifluoroacetate or ethyl trifluoroacetate.

The trialkylsilyl ester of an alkyl- or arylcarboxylic acid employed is preferably a trialkylsilyl ester having a straight-chain or branched alkyl group having 1-8 C atoms, preferably having 1-4 C atoms, where each alkyl group is independent of one another. The alkyl groups on the silicon are preferably identical. Trialkylsilyl esters of carboxylic acids are, for example, CF₃—C(O)—O—Si(CH₃)₃, CF₃—C(O)—O—Si(CH₃)₂(t-C₄H₉), CF₃—C(O)—O—Si(C₂H₅)₂(i-C₃H₇), CF₃—C(O)—O—Si(C₂H₅)₃, CF₃—C(O)—O—Si(i-C₃H₇)₃, CF₃—C(O)—O—Si-(n-C₄H₉)₃, CH₃—C(O)—O—Si(CH₃)₃, CH₃—C(O)—O—Si(CH₃)₂(n-C₄H₉), (C₂H₅)—C(O)—O—Si—(CH₃)₃, (C₂H₅)—C(O)—O—Si(C₂H₅)₃, C₂F₅—C(O)—O—Si(CH₃)₃, C₄F₉—C(O)—O—Si(CH₃)₃, C₄F₉—C(O)—O—Si(C₂H₅)₃, C₄F₉—C(O)—O—Si(CH₃)₂(n-C₄H₉), C₄F₉—C(O)—O—Si(CH₃)(n-C₄H₉)₂, C₈F₁₇—C(O)—O—Si(CH₃)₃, C₆H₅—C(O)—O—Si(CH₃)₃ or (p-NO₂)C₆H₄—C(O)—O—Si(CH₃)₃. Particular preference is given to the use of CF₃—C(O)—O—Si(CH₃)₃, C₄F₉—C(O)—O—Si(CH₃)₃, C₈F₁₇—C(O)—O—Si(CH₃)₃, C₆F₅—C(O)—O—Si(CH₃)₃ or (p-NO₂)C₆H₄—C(O)—O—Si(CH₃)₃, very particular preference is given to the use of trimethylsilyl pentafluorobenzoate (C₆F₅—C(O)—O—Si(CH₃)₃) or trimethylsilyl trifluoroacetate (CF₃—C(O)—O—Si(CH₃)₃).

The acid anhydride employed is preferably the anhydride of a straight-chain or branched alkylcarboxylic acid having 1-8 C atoms in the alkyl group, preferably having 1-4 C atoms in the alkyl group, the anhydride of an aromatic carboxylic acid, the anhydride of a sulfonic acid or the mixed anhydride of a carboxylic acid and a sulfonic acid. Suitable anhydrides are trifluoroacetic anhydride, succinic anhydride, pentafluoropropanoic anhydride, trifluoromethanesulfonic anhydride or mixed anhydrides of trifluoroacetic acid, acetic acid, propionic acid, tartaric acid, malonic acid, nicotinic acid, aminoacetic acid or benzoic acid. Particular preference is given to the use of trifluoroacetic anhydride.

Likewise suitable for use in the process according to the invention are alkyl or trialkylsilyl esters of 2,4,6-trinitrophenol (picric acid) or 2,4-dinitrophenol.

The esters or anhydrides employed are generally commercially available or can be prepared by synthetic methods as known from the literature, for example in the standard works, such as Houben-Weyl, Methoden der organischen Chemie [Methods of Organic Chemistry], Georg-Thieme-Verlag, Stuttgart, or Richard C. Larock, Comprehensive Organic Transformations, 2nd Edition, Wiley-VCH, New York, 1999. Use can also be made here of variants known per se which are not mentioned here in greater detail.

A general scheme summarises the process according to the invention:

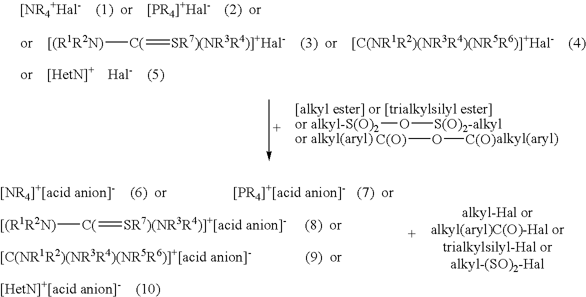

The substituents R, R¹ to R⁷ and HetN⁺ of the compounds of the formulae (1) to (10) correspond to the meanings as described above.

The reaction with alkyl esters of alkyl- or arylsulfonic acids is carried out in accordance with the invention at room temperature, i.e. generally at temperatures between 10° and 30° C. The reaction with alkyl esters of alkyl- or arylcarboxylic acids, trialkylsilyl esters of alkyl or arylsulfonic acids or alkyl- or arylcarboxylic acids is generally carried out at temperatures between 0° and 50° C., preferably 100 to 30° C., particularly preferably at room temperature. The reaction with acid anhydrides is carried out at temperatures between 20° and 180° C., preferably between 50° and 100° C. However, it can also be carried out at room temperature.

A solvent is not required. However, it is also possible to employ solvents, for example dimethoxyethane, acetonitrile, acetone, tetrahydrofuran, dimethylformamide, dimethyl sulfoxide, dioxane, propionitrile or mixtures with one another.

The reaction is carried out with an excess or equimolar amount of alkyl or trialkylsilyl esters of an alkyl- or arylsulfonic acid or an alkyl- or arylcarboxylic acid or the corresponding anhydride.

The method described is also suitable for the purification of the onium salts. This means that a corresponding ester according to the invention or an anhydride of the acid of the anion, for example trimethylsilyl trifluoromethanesulfonate, is added to the ionic liquid contaminated by halide ions, for example 1-butyl-3-methylimidazolium trifluoromethanesulfonate contaminated with 1-butyl-3-methylimidazolium chloride. The contaminant reacts away, and the halide-reduced ionic liquid is obtained.

The synthesis is also particularly suitable for heterocyclic trifluoromethanesulfonates, where a positive nitrogen in the heterocycle carries a CN group. Such compounds are excellent cyanation reagents. Exchange of the known anion tetrafluoroborate to give trifluoromethanesulfonate results in morestable compounds.

The invention therefore likewise relates to triflates of the formula (11)

HetN⁺[CF₃SO₃]⁻ (11),

where
HetN⁺ denotes a heterocyclic cation selected from the group

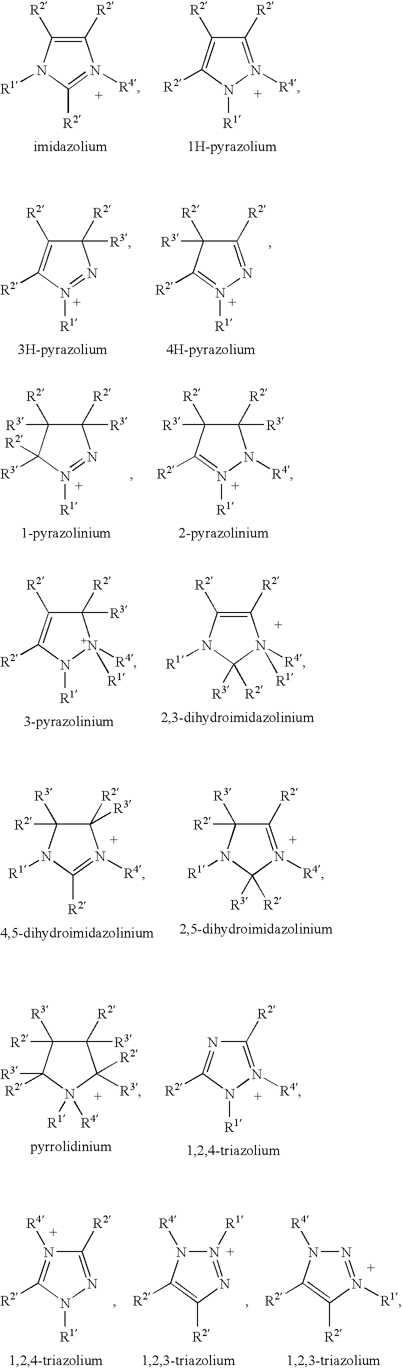

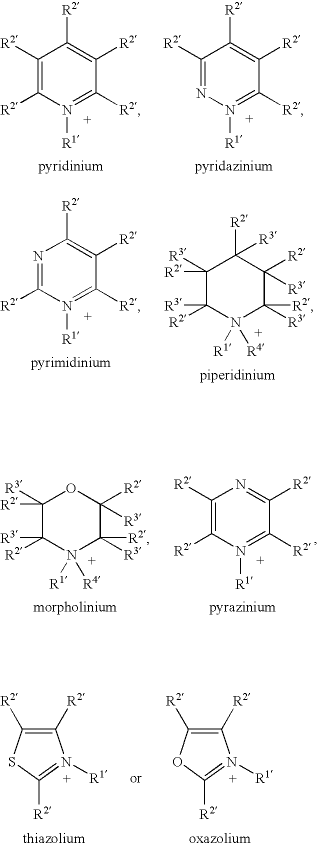

where
R^{1′} or R^{4′} denotes CN and the other substituents
R^{1′} to R^{4′} each, independently of one another, denote
hydrogen,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
dialkylamino having alkyl groups having 1-4 C atoms, but which is not bonded to the heteroatom of the heterocycle,
saturated, partially or fully unsaturated cycloalkyl having 3-7 C atoms,
which may be substituted by alkyl groups having 1-6 C atoms, or aryl-C₁-C₆-alkyl,
where one or more substituents R²′ and R³′ may be partially or fully substituted by —F,
but where R¹′ and R⁴′ are not simultaneously CN,
and where, in the substituents R¹∝ to R⁴′, one or two non-adjacent carbon atoms which are not bonded directly to the heteroatom and are not in the ω-position may be replaced by atoms and/or atom groups selected from the group —O—, —S—, —S(O)— or —SO₂—.

In accordance with the invention, suitable substituents R^{1′} to R^{4′} of compounds of the formula (5), besides hydrogen, are preferably: CN, C₁- to C₂₀-, in particular C₁- to C₁₂-alkyl groups, and saturated or unsaturated, i.e. also aromatic, C₃- to C₇-cycloalkyl groups, which may be substituted by C₁- to C₆-alkyl groups, in particular phenyl or aryl-C₁-C₆-alkyl.

One substituent R^{1′} or R^{4′} is by definition CN. The second substituent R^{1′} or R^{4′} is particularly preferably methyl, ethyl, isopropyl, propyl, butyl, sec-butyl, pentyl, hexyl, octyl, decyl, cyclohexyl, phenyl, phenylpropyl or benzyl, very particularly preferably methyl, ethyl, n-butyl or hexyl.

The substituent R^{2′} or R^{3′} is in each case, independently of one another, in particular hydrogen, dimethylamino, diethylamino, methyl, ethyl, isopropyl, propyl, butyl, sec-butyl, cyclohexyl, phenyl or benzyl. R^{2′} is particularly preferably hydrogen, methyl, ethyl, isopropyl, propyl, butyl, sec-butyl or dimethylamino. R^{2′} and R^{3′} are very particularly preferably each, independently of one another, hydrogen.

HetN⁺ of the formula (11) is preferably

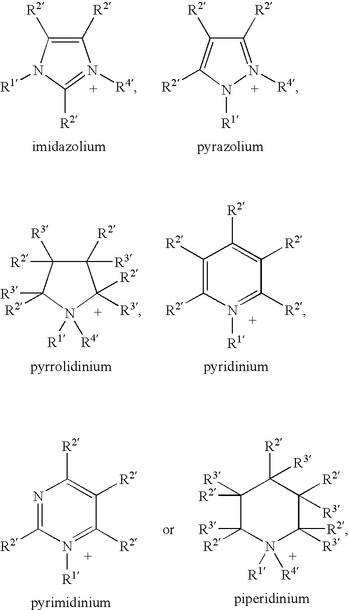

where the substituents R^{1′} to R^{4′} each, independently of one another, have a meaning described above.

HetN⁺ is particularly preferably imidazolium, pyrrolidinium or pyridinium, as defined above, where the substituents R^{1′} to R^{4′} each, independently of one another, have a meaning described above, very particularly preferably pyridinium.

Even without further comments, it is assumed that a person skilled in the art will be able to utilise the above description in the broadest scope. The preferred embodiments and examples should therefore merely be regarded as descriptive disclosure which is absolutely not limiting in any way.

It goes without saying for the person skilled in the art that substituents in the compounds mentioned above and below, such as, for example, H, N, O, Cl or F, can be replaced by the corresponding isotopes.

The NMR spectra were measured on solutions in deuterated solvents at 20° C. on a Bruker ARX 400 spectrometer with a 5 mm ¹H/BB broadband head with deuterium lock, unless indicated in the examples. The measurement frequencies of the various nuclei are: ¹H: 400.13 MHz and ¹⁹F: 276.50 MHz. The referencing method is indicated separately for each spectrum or each data set.

### EXAMPLE 1

### Synthesis of 1-hexyl-3-methylimidazolium trifluoromethanesulfonate (triflate)

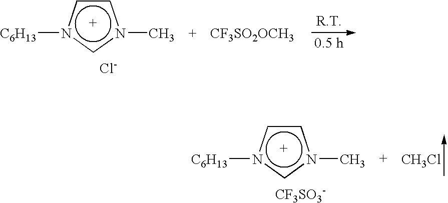

A mixture of 1.80 g (8.88 mmol) of 1-hexyl-3-methylimidazolium chloride and 1.52 g (9.26 mmol) of methyl triflate, CF₃SO₂OCH₃, is stirred at room temperature for 30 minutes. NMR measurements show the completeness of the reaction. The residue is dried for 30 minutes in vacuo at 13.3 Pa and 120° C. (oil-bath temperature), giving 2.80 g of 1-hexyl-3-methylimidazolium triflate in approximately quantitative yield.

¹H NMR (reference: TMS; CD₃CN), ppm: 0.87 m (CH₃); 1.29 m (3CH₂); 1.81 m (CH₂); 3.82 s (CH₃); 4.11 t (CH₂); 7.35 d,d (CH); 7.39 d,d (CH); 8.55 br. s. (CH); ³J_{H,H}=6.9 Hz; J_{H,H}=1.5 Hz.

¹⁹F NMR (reference: CCl₃F—internal; CD₃CN), ppm: −78.1 s (CF₃).

### EXAMPLE 2

### Synthesis of 1-butyl-3-methylimidazolium triflate

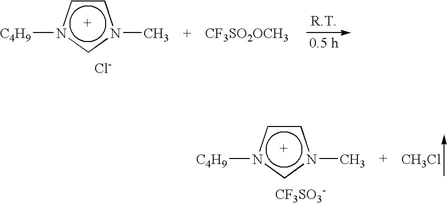

1.04 g (5.95 mmol) of 1-butyl-3-methylimidazolium chloride are reacted with 1.67 g (10.2 mmol) of methyl triflate analogously to Example 1, giving 1.71 g of 1-butyl-3-methylimidazolium triflate in approximately quantitative yield.

¹H NMR (reference: TMS; CD₃CN), ppm: 0.92 t (CH₃); 1.31 m (CH₂);

1.80 m (CH₂); 3.82 S(CH₃); 4.12 t (CH₂); 7.34 d,d (CH); 7.38 d,d (CH); 8.51 br. s. (CH); ³J_{H,H}=7.4 Hz; ³J_{H,H}=7.1 Hz; J_{H,H}=1.7 Hz.

¹⁹F NMR (reference: CCl₃F—internal; CD₃CN), ppm: −78.11 s (CF₃).

### EXAMPLE 3

### Synthesis of 1-ethylpyridinium triflate

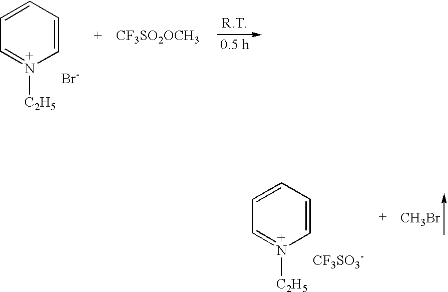

1.56 g (8.29 mmol) of 1-ethylpyridinium bromide are reacted with 1.67 g (10.2 mmol) of methyl triflate analogously to Example 1, giving 2.13 g of 1-ethylpyridinium triflate in approximately quantitative yield.

¹H NMR (reference: TMS; CD₃CN), ppm: 1.58 t (CH₃); 4.59 q (CH₂); 8.02 m (2CH); 8.49 t,t (CH); 8.78 d (2CH); ³J_{H,H}=7.3 Hz; ³J_{H,H}=7.6 Hz; ³J_{H,H}=6.1 Hz; ⁴J_{H,H}=1.2 Hz.

¹⁹F NMR (reference: CCl₃F—internal; CD₃CN), ppm: −78.0 s (CF₃).

### EXAMPLE 4

### Synthesis of 1-butylpyridinium triflate

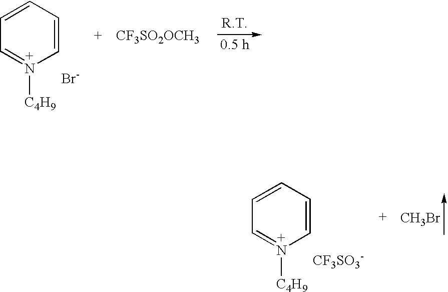

4.31 g (19.9 mmol) of 1-butylpyridinium bromide are reacted with 4.41 g (26.9 mmol) of methyl triflate analogously to Example 1, giving 5.68 g of 1-butylpyridinium triflate in approximately quantitative yield.

¹H NMR (reference: TMS; CD₃CN), ppm: 0.93 t (CH₃); 1.35 m (CH₂);

1.93 m (CH₂); 4.53 t (CH₂); 8.02 m (2CH); 8.50 t (CH); 8.74 d (20H); ³J_{H,H}=7.4 Hz; ³J_{H,H}=7.6 Hz; ³J_{H,H}=7.9 Hz; ³J_{H,H}=6.1 Hz.

¹⁹F NMR (reference: CCl₃F—internal; CD₃CN), ppm: −78.0 s (CF₃).

### EXAMPLE 5

### Synthesis of trihexyltetradecylphosphonium triflate

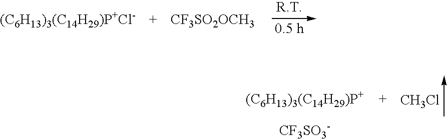

1.75 g (3.37 mmol) of trihexyltetradecylphosphonium chloride are reacted with 2.59 g (15.8 mmol) of methyl triflate for 30 minutes analogously to Example 1, giving 2.13 g of trihexyltetradecylphosphonium triflate in approximately quantitative yield.

¹H NMR (reference: TMS; CD₃CN), ppm: 0.84-0.94 m (4CH₃), 1.24-1.37 m (16CH₂), 1.37-1.57 m (8CH₂), 1.99-2.09 m (4CH₂).

¹⁹F NMR (reference: CCl₃F—internal; CD₃CN), ppm: −78.0 s (CF₃).

³¹P {¹H} NMR (reference: 85% H₃PO₄—external; CD₃CN), ppm: 33.5.

### EXAMPLE 6

### Synthesis of 1-ethylpyridinium triflate

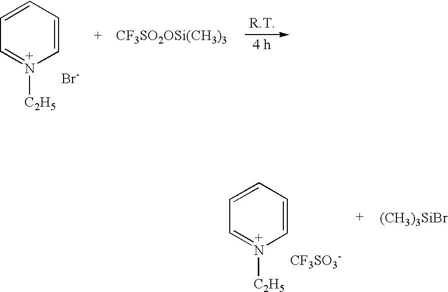

A mixture of 1.85 g (9.84 mmol) of 1-ethylpyridinium bromide and 2.54 g (11.4 mmol) of trimethylsilyl triflate, CF₃SO₂OSi(CH₃)₃, is stirred at room temperature for 4 hours. NMR measurements show the completeness of the reaction. The residue is dried for 30 minutes in vacuo at 13.3 Pa and 120° C. (oil-bath temperature), giving 2.53 g of 1-ethylpyridinium triflate in approximately quantitative yield.

The NMR spectra correspond to those from Example 3.

### EXAMPLE 7

### Synthesis of 1-butylpyridinium triflate

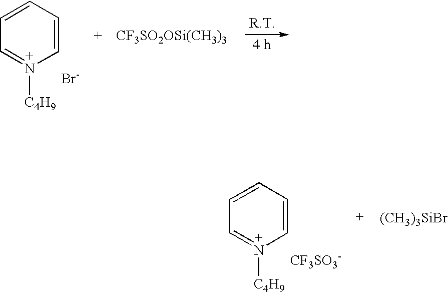

1.42 g (6.57 mmol) of 1-butylpyridinium bromide are reacted with 1.73 g (7.78 mmol) of trimethylsilyl triflate analogously to Example 6, giving 1.87 g of 1-butylpyridinium triflate in approximately quantitative yield.

The NMR spectra correspond to those from Example 4.

### EXAMPLE 8

### Synthesis of 1-butyl-3-methylimidazolium triflate

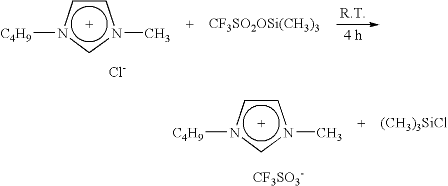

1.67 g (9.56 mmol) of 1-butyl-3-methylimidazolium chloride are reacted with 2.36 g (10.6 mmol) of trimethylsilyl triflate analogously to Example 6, giving 2.75 g of 1-butyl-3-methylimidazolium triflate in approximately quantitative yield.

The NMR spectra correspond to those from Example 2.

### EXAMPLE 9

### Synthesis of 1-hexyl-3-methylimidazolium triflate

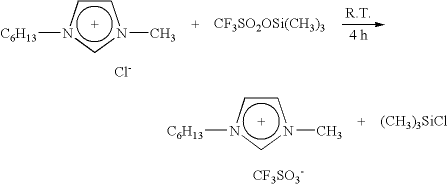

1.81 g (8.93 mmol) of 1-hexyl-3-methylimidazolium chloride are reacted with 2.62 g (11.79 mmol) of trimethylsilyl triflate analogously to Example 6, giving 2.81 g of 1-hexyl-3-methylimidazolium triflate in approximately quantitative yield.

The NMR spectra correspond to those from Example 1.

### EXAMPLE 10

### Synthesis of Trihexyltetradecylphosphonium Triflate

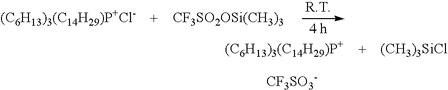

1.64 g (3.16 mmol) of trihexyltetradecylphosphonium chloride are reacted with 0.91 g (4.09 mmol) of trimethylsilyl triflate analogously to Example 6, giving 2.00 g of trihexyltetradecylphosphonium triflate in approximately quantitative yield.

The NMR spectra correspond to those from Example 5.

### EXAMPLE 11

Analogously to Example 6,

1-methylimidazolium chloride is reacted with trimethylsilyl triflate to give
1-methylimidazolium triflate;
1-butylimidazolium chloride is reacted with trimethylsilyl triflate to give
1-butylimidazolium triflate;
   1,3-dimethylimidazolium chloride is reacted with trimethylsilyl triflate to give
1,3-dimethylimidazolium triflate;
   1-ethyl-3-methylimidazolium chloride is reacted with trimethylsilyl triflate to give
1-ethyl-3-methylimidazolium triflate;
   1-butyl-3-ethylimidazolium chloride is reacted with trimethylsilyl triflate to give
1′-butyl-3-ethylimidazolium triflate;
   1-methyl-3-pentylimidazolium chloride is reacted with trimethylsilyl triflate to give
1-methyl-3-pentylimidazolium triflate;
   1-octyl-3-methylimidazolium chloride is reacted with trimethylsilyl triflate to give
1-octyl-3-methylimidazolium triflate;
   1-benzyl-3-methylimidazolium chloride is reacted with trimethylsilyl triflate to give
1-benzyl-3-methylimidazolium triflate;
   1-phenylpropyl-3-methylimidazolium chloride is reacted with trimethylsilyl triflate to give
1-phenylpropyl-3-methylimidazolium triflate;
   1-ethyl-2,3-dimethylimidazolium chloride is reacted with trimethylsilyl triflate to give
1-ethyl-2,3-dimethylimidazolium triflate;
   1-butyl-2,3-dimethylimidazolium chloride is reacted with trimethylsilyl triflate to give
1-butyl-2,3-dimethylimidazolium triflate;
   1-hexyl-2,3-dimethylimidazolium chloride is reacted with trimethylsilyl triflate to give
1-hexyl-2,3-dimethylimidazolium triflate;
   1-hexylpyridinium bromide is reacted with trimethylsilyl triflate to give
1-hexylpyridinium triflate;
   1-butyl-3-methylpyridinium bromide is reacted with trimethylsilyl triflate to give
1-butyl-3-methylpyridinium triflate;
   1-butyl-3-ethylpyridinium bromide is reacted with trimethylsilyl triflate to give
   1-butyl-3-ethylpyridinium triflate or
   methyltrioctylammonium chloride is reacted with trimethylsilyl triflate to give methyltrioctylammonium triflate.

### EXAMPLE 12

### Synthesis of 1-butyl-3-methylimidazolium methylsulfonate

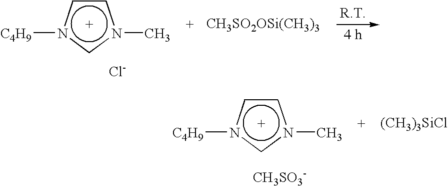

2.06 g (11.8 mmol) of 1-butyl-3-methylimidazolium chloride are reacted with 1.99 g (11.8 mmol) of trimethylsilyl methanesulfonate, CH₃SO₃Si(CH₃)₃, analogously to Example 6, giving 2.72 g of 1-butyl-3-methylimidazolium methylsulfonate in approximately quantitative yield.

¹H NMR (reference: TMS; CD₃CN), ppm: 0.91 t (CH₃); 1.30 m (CH₂);

1.80 m (CH₂); 2.47 s (CH₃); 3.87 S(CH₃); 4.18 t (CH₂); 7.48 d,d (CH); 7.51 d,d (CH); 9.23 br. s. (CH); ³J_{H,H}=7.4 Hz; J_{H,H}=7.2 Hz; J_{H,H}=1.7 Hz.

### EXAMPLE 13

### Synthesis of 1-butyl-3-methylimidazolium benzenesulfonate

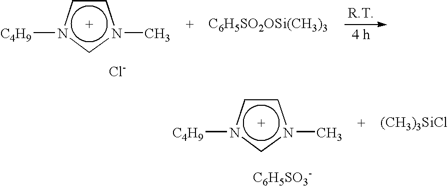

1.57 g (8.99 mmol) of 1-butyl-3-methylimidazolium chloride are reacted with 2.09 g (9.07 mmol) of trimethylsilyl benzenesulfonate, C₆H₅SO₃Si(CH₃)₃, analogously to Example 6. Drying in vacuo at an oilbath temperature of 60° C. gives 2.66 g of 1-butyl-3-methylimidazolium benzenesulfonate in approximately quantitative yield.

¹H NMR (reference: TMS; CD₃CN), ppm: 0.89 t (CH₃); 1.27 m (Ch₂);

1.76 m (CH₂); 3.82 s (CH₃); 4.12 t (CH₂); 7.32-7.39 (3H, C₆H₅); 7.39 d,d (CH); 7.43 d,d (CH); 7.70-7.79 (2H, C₆H₅); 9.01 br. s. (CH); ³J_{H,H}=7.4 Hz; ³J_{H,H}=7.3 Hz; J_{H,H}=1.8 Hz.

### EXAMPLE 14

### Synthesis of 1-butyl-3-methylimidazolium trifluoroacetate

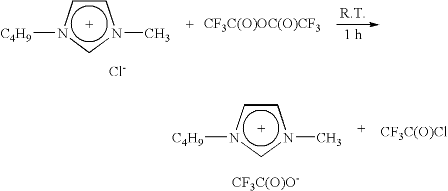

A mixture of 2.42 g (13.85 mmol) of 1-butyl-3-methylimidazolium chloride and 3.21 g (15.28 mmol) of trifluoroacetic anhydride, CF₃C(O)OC(O)CF₃, is stirred at room temperature for 3 hours. NMR measurements show the completeness of the reaction. The residue is dried for 30 minutes in vacuo at 13.3 Pa and 60° C. (oil-bath temperature), giving 3.49 g of 1-butyl-3-methylimidazolium trifluoroacetate in approximately quantitative yield.

¹H NMR (reference: TMS; CD₃CN), ppm: 0.89 t (CH₃); 1.27 m (CH₂);

1.78 m (CH₂); 3.34 s (CH₃); 4.15 t (CH₂); 7.44 d,d (CH); 7.49 d,d (CH), 9.24 br. s. (CH); ³J_{H,H}=7.4 Hz; J_{H,H}=1.7 Hz.

¹⁹F NMR (reference: CCl₃F—internal; CD₃CN), ppm: −74.4 s (CF₃).

### EXAMPLE 15

Analogously to Example 14,

1,3-dimethylimidazolium chloride is reacted with trifluoroacetic anhydride to give
1,3-dimethylimidazolium trifluoroacetate;
1-butyl-1-methylpyrrolidinium chloride is reacted with trifluoroacetic anhydride to give
1-butyl-1-methylpyrrolidinium trifluoroacetate or
   methyltrioctylammonium chloride is reacted with trifluoroacetic anhydride to give methyltrioctylammonium trifluoroacetate.

### EXAMPLE 16

### Synthesis of 1-ethyl-3-methylimidazolium p-toluenesulfonate (tosylate)

### a) Synthesis of 1-ethyl-3-methylimidazolium bromide

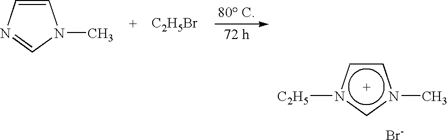

111.43 g (1.36 mol) of methylimidazole and 160 g (1.47 mot) of bromoethane are mixed, and 400 ml of isopropanol are subsequently added. The reaction mixture is heated for 72 hours with stirring, during which the oil-bath temperature is 80° C. Isopropanol is then removed by distillation, and the residue is dried for two hours in vacuo at 13.3 Pa and an oil-bath temperature of 100° C., giving 258.6 g of 1-ethyl-3-methylimidazolium bromide, corresponding to a yield of 99.7%.

M.p.: 73-74° C.

¹H NMR (reference: TMS; CD₃CN), ppm: 1.44 t (CH₃); 3.88 s (CH₃); 4.23 q (CH₂); 7.49 m (CH); 7.56 m (CH); 9.45 br. s. (CH); ³J_{H,H}=7.2 Hz.

### b) Synthesis of 1-ethyl-3-methylimidazolium tosylate

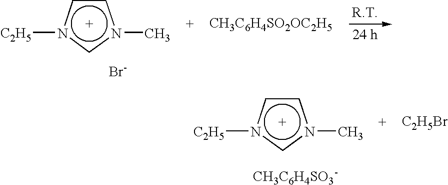

45.5 g (0.227 mol) of molten ethyl tosylate (p-CH₃C₆H₄SO₂OC₂H₅, m.p. 32-34° C.) are added to 43.41 g (0.227 mol) of 1-ethyl-3-methylimidazolium bromide. The reaction mixture is stirred at room temperature for 24 hours until the bromide salt has completely dissolved. The liquid product is subsequently dried for one hour in vacuo at 13.3 Pa and an oil-bath temperature of 60° C., giving 64.1 g of 1-ethyl-3-methylimidazolium tosylate, corresponding to an approximately quantitative yield.

¹H NMR (reference: TMS; CD₃CN), ppm: 1.35 t (CH₃); 2.29 s (CH₃); 3.78 s (CH₃); 4.11 q (CH₂); 7.13 d (2CH, A); 7.60 d (2CH, B); 7.42 m (CH); 7.49 m (CH); 9.10 br. s. (CH); ³J_{H,H}=7.3 Hz; ³J_{H(A),H(B)}=8.1 Hz.

### EXAMPLE 17

Analogously to Example 16,

1-methylimidazolium chloride is reacted with trimethylsilyl (p-toluene)sulfonate to give
1-methylimidazolium p-toluenesulfonate;
   1-butylimidazolium chloride is reacted with trimethylsilyl (p-toluene)sulfonate to give
1-butylimidazolium p-toluenesulfonate;
   1-butyl-3-methylimidazolium chloride is reacted with trimethylsilyl (p-toluene)sulfonate to give
1-butyl-3-methylimidazolium p-toluenesulfonate;
   1-butyl-2,3-dimethylimidazolium chloride is reacted with trimethylsilyl (p-toluene)sulfonate to give
1-butyl-2,3-dimethylimidazolium p-toluenesulfonate or
   triisobutylmethylphosphonium bromide is reacted with trimethylsilyl (p-toluene)sulfonate to give
   triisobutylmethylphosphonium p-toluenesulfonate.

### EXAMPLE 18

### Synthesis of N,N,N′,N′-tetramethyl-N″-ethylguanidinium trifluoromethanesulfonate

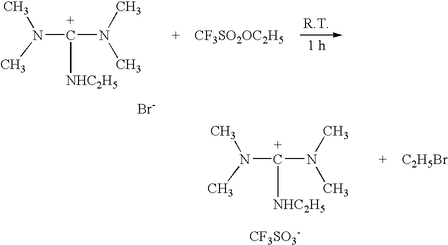

A mixture of 2.38 g (10.62 mmol) of N,N,N′N′-tetramethyl-N″-ethylguanidinium bromide and 1.95 g (10.95 mmol) of ethyl trifluoromethanesulfonate is stirred at room temperature for one hour. An NMR measurement shows the completeness of the reaction. The residue is subsequently dried for 2 hours in a vacuum of 13.3 Pa and at an oil-bath temperature of 60° C., giving 3.11 g of N,N,N′,N′-tetramethyl-N″-ethylguanidinium trifluoromethanesultfonate as a liquid, corresponding to an approximately quantitative yield.

¹H NMR (reference: TMS; CD₃CN), ppm: 1.13 t (CH₃); 2.87 br.s; 2.89 br.s;

2.92 S (4CH₃); 3.22 m (CH₂); 6.42 br.s (NH); ³J_{H,H}=7.1 Hz.

¹⁹F NMR (reference: CCl₃F—internal; CD₃CN), ppm: −78.0 s (CF₃).

Analogously thereto,

N,N,N′,N′,N″-pentamethyl-N″-propylguanidinium chloride is reacted with methyl or ethyl triflate to give
N,N,N′,N′,N″-pentamethyl-N″-propylguanidinium triflate;
   hexamethylguanidinium chloride is reacted with methyl or ethyl triflate to give
hexamethylguanidinium triflate or
   S-ethyl-N,N,N′,N′-tetramethylisothiouronium iodide is reacted with methyl or ethyl triflate to give
S-ethyl-N,N,N′,N′-tetramethylisothiouronium triflate.

### EXAMPLE 19

### Synthesis of 1-cyano-4-dimethylaminopyridinium trifluoromethanesulfonate

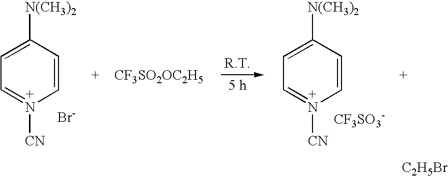

A mixture of 1.88 g (8.24 mmol) of 1-cyano-4-dimethylaminopyridinium bromide and 1.93 g (10.83 mmol) of ethyl trifluoromethanesulfonate is stirred at room temperature for 5 hours. The volatile compounds are subsequently removed in vacuo, and the residue is dried for 2 hours at room temperature in a vacuum of 13.3 Pa, giving 2.40 g of 1-cyano-4-dimethylaminopyridinium trifluoromethanesulfonate, corresponding to a yield of 98%.

M.p.: 75-77° C.

¹H NMR (reference: TMS; CD₃CN), ppm: 3.33 s (2CH₃); 7.02 d,m (2CH, A);

8.09 d,m (2CH, B); ³J_{H(A),H(B)}=8.1 Hz.

¹⁹F NMR (reference: CCl₃F—internal; CD₃CN), ppm: −78.02 s (CF₃).

¹³C NMR (reference: TMS; CD₃CN), ppm: 42.2 q,q [N(CH₃)₂]; 107.6 m (CN); 109.8 d,m (2CH); 141.5 d,m (2CH); 158.0 m (C); ¹J_{C,H}=195 Hz; ¹J_{C,H}=175 Hz; ¹J_{C,H}=142 Hz; ³J_{C,H}=3.3 Hz.

Raman spectrum: 2272.9 cm⁻¹ (CN).

Elemental analysis C₉H₁₀F₃N₃O₃S (molecular weight 297.25):

found: C, 36.00%; H, 3.22%; N, 13.92%; S, 9.84%.

calculated: C, 36.37%; H 3.39%; N, 14.14%; S, 10.79%.

### EXAMPLE 20

### Synthesis of 1-ethyl-2,3-dimethylimidazolium p-toluenesulfonate

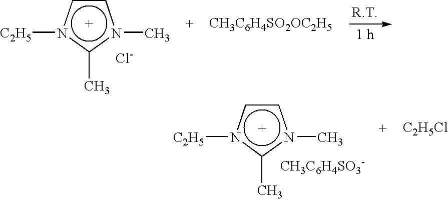

2.65 g (13.23 mmol) of molten ethyl tosylate (melting point 32-34° C.) are added to 2.08 g (12.95 mmol) of 1-ethyl-2,3-dimethylimidazolium bromide. The reaction mixture is stirred at room temperature for 1 hour and dried in vacuo at 13.3 Pa and an oil-bath temperature of 110-112° C., giving 3.83 g of 1-ethyl-2,3-dimethylimidazolium p-toluenesulfonate. The yield is approximately quantitative.

¹H NMR (reference: TMS; CD₃CN), ppm: 1.33 t (CH₃); 2.32 s (CH₃); 2.48 s (CH₃); 3.68 s (CH₃); 4.07 q (CH₂); 7.13 d (2CH, A); 7.47 d (2CH, B); 7.36 m (2CH); ³J_{H,H}=7.3 Hz; ³J_{H(A),H(B)}=8.0 Hz.

### EXAMPLE 21

### Synthesis of 1-ethyl-1-methylpyrrolidinium triflate

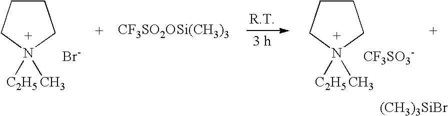

A mixture of 2.36 g (12.15 mmol) of 1-ethyl-1-methylpyrrolidinium bromide and 2.70 g (12.15 mmol) of trimethylsilyl triflate is stirred at room temperature for 3 hours. All volatile products are removed over the course of one hour in vacuo at 13.3 Pa and 60° G, giving 3.19 g of 1-ethyl-1-methylpyrrolidinium triflate. The yield is approximately quantitative.

M.p.: 94-95° C.

¹H NMR (reference: TMS; CD₃CN), ppm: 1.32 μm (CH₃); 2.15 m (2CH₂);

2.95 s (CH₃); 3.35 q (CH₂); 3.42 m (2CH₂); ³J_{H,H}=7.3 Hz.

¹⁹F NMR (reference: CCl₃F-internal; CD₃CN), ppm: −78.03 s (CF₃).

Analogously thereto,

1-butyl-1-methylpyrrolidinium chloride is reacted with trimethylsilyl triflate to give
1-butyl-1-methylpyrrolidinium triflate.

### EXAMPLE 22

### Synthesis of 1-methylimidazolium triflate

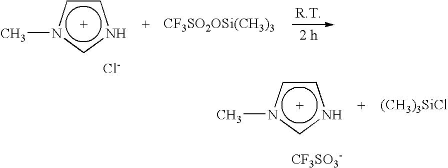

A mixture of 2.23 g (18.80 mmol) of 1-methylimidazolium chloride and 4.61 g (20.74 mmol) of trimethylsilyl triflate is stirred at room temperature for 2 hours. NMR measurements show the completeness of the reaction. The residue is dried for 1 hour in vacuo at 13.3 Pa and an oil-bath temperature of 60° C., giving 4.36 g of 1-methylimidazolium triflate. The yield is approximately quantitative.

M.p.: 90-91° C.

¹H NMR (reference: TMS; CD₃CN), ppm: 3.86 s (CH₃); 7.38 br.s (2CH); 8.54 br.s (CH); 12.30 br. s (NH).

¹⁹F NMR (reference: CCl₃F—internal; CD₃CN), ppm: —78.1 s (CF₃).

### EXAMPLE 23

### Synthesis of 1-ethyl-3-methylimidazolium trifluoroacetate

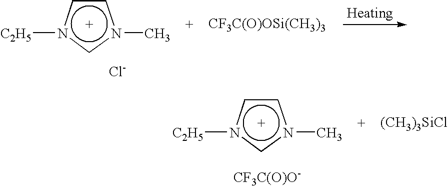

A mixture of 19.14 g (131 mmol) of 1-ethyl-3-methylimidazolium chloride and 72.62 g (390 mmol) of trimethylsilyl trifluoroacetate, CF₃C(O)OSi(CH₃)₃, is heated and refluxed for three hours. Trimethylchlorosilane, (CH₃)₃SiCl, which forms during the reaction and excess trimethylsilyl trifluoroacetate are removed by distillation over a column. The residue is pumped over within 30 minutes at 13.3 Pa and at a bath temperature of 80° C., giving 29.25 g of 1-ethyl-3-methylimidazolium trifluoroacetate. The yield is virtually quantitative.

¹H NMR (reference: TMS; CD₃CN), ppm: 1.41 t (CH₃); 3.82 s (CH₃); 4.16 q (CH₂); 7.38 d,d (CH); 7.44 d,d (CH); 8.79 br. s. (CH); ³J_{H,H}=7.3 Hz; J_{H,H}=1.7 Hz.

¹⁹F NMR (reference: CCl₃F—internal; CD₃CN), ppm: −74.5 s (CF₃).

1. Process for the preparation of onium salts with alkyl- or arylsulfonate anions or alkyl- or arylcarboxylate anions by reaction of an onium halide with an alkyl or trialkylsilyl ester of an alkyl- or arylsulfonic acid or alkyl- or arylcarboxylic acid or anhydrides thereof.

2. Process according to claim 1, characterised in that, for the synthesis of alkyl- or arylsulfonate salts, an onium halide is reacted with a trialkylsilyl ester of an alkyl- or arylsulfonic acid.

3. Process according to claim 1, characterised in that, for the synthesis of alkyl- or arylcarboxylate salts, an onium halide is reacted with an anhydride of an alkyl- or arylcarboxylic acid.

4. Process according to claim 1, characterised in that the halide is an ammonium halide, phosphonium halide, thiouronium halide, guanidinium halide or a halide with a heterocyclic cation.

5. Process according to claim 1, characterised in that the halide conforms to the formula (1)
[NR₄]⁺Hal⁻ (1),
where
Hal denotes Cl, Br or I and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more R may be partially or fully substituted by —F, but where all four or three R must not be fully substituted by F,
and where, in the R, one or two non-adjacent carbon atoms which are not in the α- or ω-position may be replaced by atoms and/or atom groups selected from the group —O—, —S—, —S(O)— or —SO₂—.

6. Process according to claim 1, characterised in that the halide conforms to the formula (2)
[PR₄]⁺Hal⁻ (2),
where
Hal denotes Cl, Br or I and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more R may be partially or fully substituted by —F, but where all four or three R must not be fully substituted by F,
and where, in the R, one or two non-adjacent carbon atoms which are not in the α- or ω-position may be replaced by atoms and/or atom groups selected from the group —O—, —S—, —S(O)— or —SO₂—.

7. Process according to claim 1, characterised in that the halide conforms to the formula (3)
[(R¹R²N)—C(═SR⁷)(NR³R⁴)]⁺Hal⁻ (3),
where
Hal denotes Cl, Br or I and
R¹ to R⁷ each, independently of one another, denote
hydrogen or CN, where hydrogen is excluded for R⁷,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more of the substituents R¹ to R⁷ may be partially or fully substituted by —F, but where all substituents on an N atom must not be fully substituted by F,
where the substituents R¹ to R⁷ may be connected to one another in pairs by a single or double bond
and where, in the substituents R¹ to R⁷, one or two non-adjacent carbon atoms which are not bonded directly to the heteroatom and are not in the ω-position may be replaced by atoms and/or atom groups selected from the group —O—, —S—, —S(O)— or —SO₂—.

8. Process according to claim 1, characterised in that the halide conforms to the formula (4)
[C(NR¹R²)(NR³R⁴)(NR⁵R⁶)]⁺Hal⁻ (4),
where
Hal denotes Cl, Br or I and
R¹ to R⁶ each, independently of one another, denote hydrogen or CN,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more of the substituents R¹ to R⁶ may be partially or fully substituted by —F, but where all substituents on an N atom must not be fully substituted by F,
where the substituents R¹ to R⁶ may be connected to one another in pairs by a single or double bond
and where, in the substituents R¹ to R⁶, one or two non-adjacent carbon atoms which are not bonded directly to the heteroatom and are not in the ω-position may be replaced by atoms and/or atom groups selected from the group —O—, —S—, —S(O)— or —SO₂—.

9. Process according to claim 1, characterised in that the halide conforms to the formula (5)
[HetN]⁺Hal⁻ (5),
where
Hal denotes Cl, Br or I and
HetN⁺ denotes a heterocyclic cation selected from the group 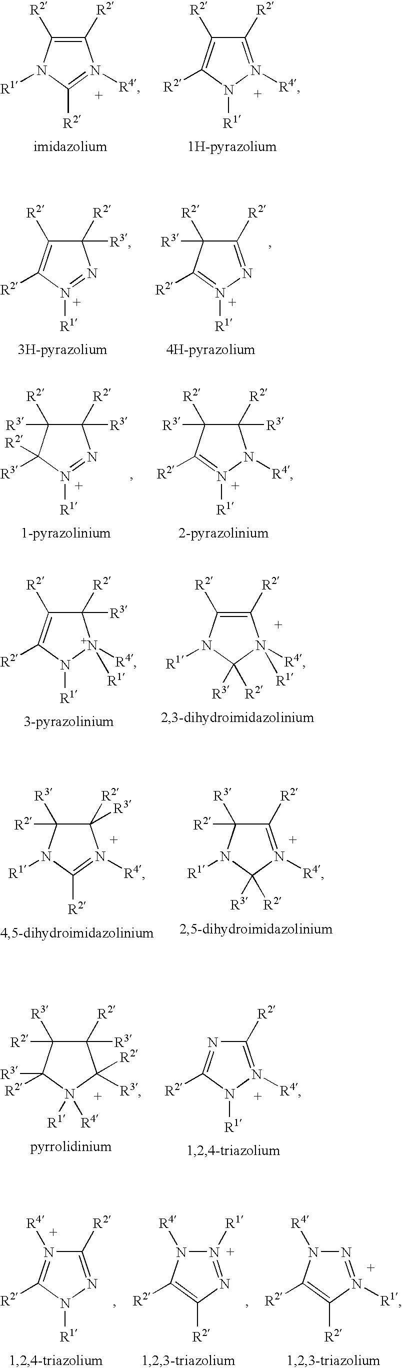 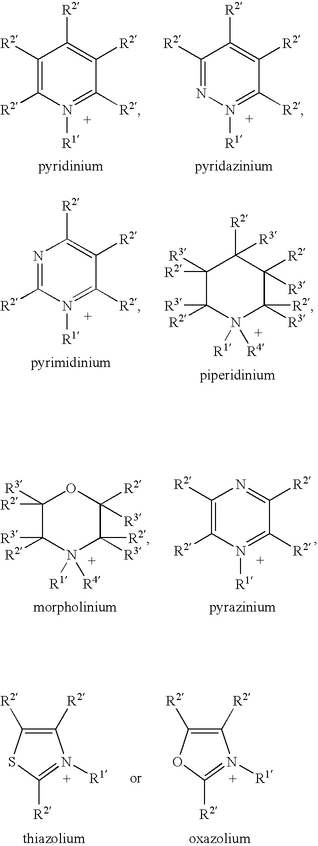
where the substituents
R¹′ to R⁴′ each, independently of one another, denote hydrogen or CN,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
dialkylamino having alkyl groups having 1-4 C atoms, but which is not bonded to the heteroatom of the heterocycle,
saturated, partially or fully unsaturated cycloalkyl having 3-7 C atoms, which may be substituted by alkyl groups having 1-6 C atoms,
or aryl-C₁-C₆-alkyl,
where the substituents R¹′ and R⁴′ may be partially or fully substituted by F, but
where R^{1′} and R^{4′} are not simultaneously CN or must not simultaneously be fully substituted by F,
where the substituents R^{2′} and R^{3′} may be partially or fully substituted by halogens or partially substituted by NO₂ or CN
and where, in the substituents R¹′ to R⁴′, one or two non-adjacent carbon atoms which are not bonded directly to the heteroatom and are not in the ω-position may be replaced by atoms and/or atom groups selected from the group —O—, —S—, —S(O)— or —SO₂—.

10. Process according to claim 1, characterised in that the reaction is carried out without a solvent.

11. Use of the process according to claim 1 for the purification of ionic liquids with alkyl- or arylsulfonate anions or alkyl- or arylcarboxylate anions which are contaminated by onium halides.

12. Trifluoromethanesulfonates of the formula (11)
HetN⁺[CF₃SO₃]⁻ (11),
where
HetN⁺ denotes a heterocyclic cation selected from the group 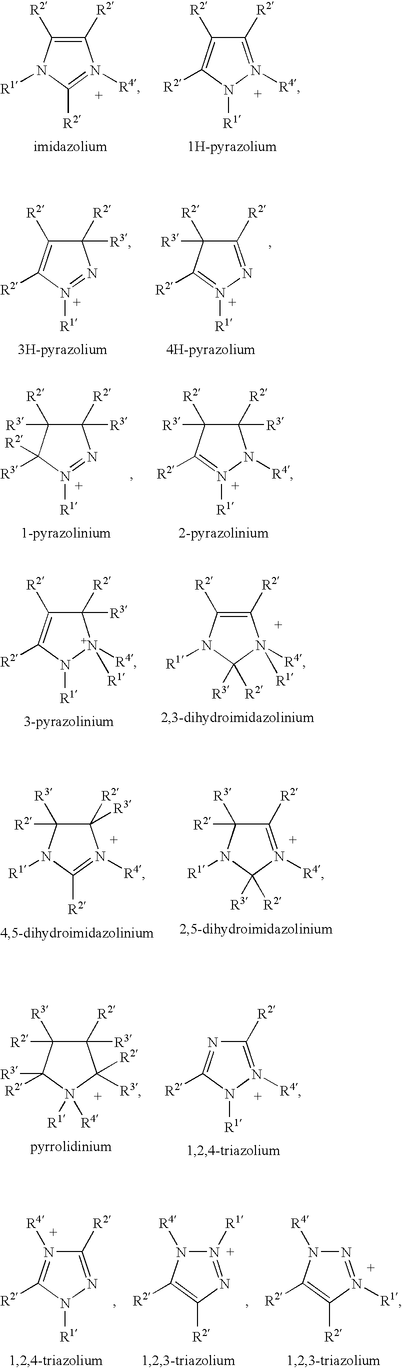 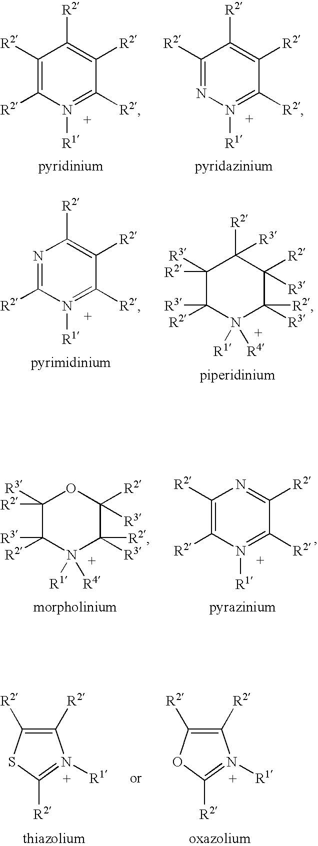
where
R^{1′} or R^{4′} denotes CN and the other substituents
R¹′ to R⁴′ each, independently of one another, denote hydrogen,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
dialkylamino having alkyl groups having 1-4 C atoms, but which is not bonded to the heteroatom of the heterocycle,
saturated, partially or fully unsaturated cycloalkyl having 3-7 C atoms, which may be substituted by alkyl groups having 1-6 C atoms,
or aryl-C₁-C₆-alkyl,
where one or more substituents R²′ and R³′ may be partially or fully substituted by —F,
but where R¹′ and R⁴′ are not simultaneously CN, and where, in the substituents R¹′ to R⁴′, one or two non-adjacent carbon atoms which are not bonded directly to the heteroatom and are not in the ω-position may be replaced by atoms and/or atom groups selected from the group —O—, —S—, —S(O)— or —SO₂—.
